(19) United States
(12) Patent Application Publication
(10) Pub. No.: US 2014/0350496 A1
(43) Pub. Date: Nov. 27, 2014

(54) SET COMPRISING WOUND DRESSING AND WOUND FILLER

(71) Applicant: Birgit Riesinger, Munster (DE)

(72) Inventor: Birgit Riesinger, Munster (DE)

(21) Appl. No.: 14/297,362

(22) Filed: Jun. 5, 2014

Related U.S. Application Data
(63) Continuation of application No. PCT/EP2012/074836, filed on Dec. 7, 2012.

(30) Foreign Application Priority Data
Dec. 9, 2011 (DE) 10 2011 056 245.1

(51) Int. Cl. A61F 13/00

(52) U.S. Cl. CPC A61F 13/00008; A61F 13/00012; A61F 13/00068; A61F 13/00042
USPC 604/319; 604/367; 604/368

(57) ABSTRACT

The invention relates to a set of wound care articles, comprising at least one first planar wound care article comprising hydroactive polymers and at least one second wound care article comprising hydroactive polymers, wherein the second and optionally also any additional wound care articles have a lower liquid retention and/or a lower liquid absorption capacity than the first wound care article.

### CROSS-REFERENCE TO RELATED APPLICATIONS

The present application claims priority from and is a continuation from PCT Application No. PCT/EP2012/074836, filed Dec. 7, 2012, which claims priority from German Patent Application No. DE 10 2011 056 245.1, filed Dec. 9, 2011, all herein incorporated by reference in their entireties.

### BACKGROUND

The present invention relates to a wound care article set as classified in the preamble of claim 1.

Wound care articles comprising a body absorbing wound exudates are known from the prior art, for example from commonly assigned DE10059439. The body in question comprises an essentially flat absorbent-material portion consisting of an imbibing fibrous nonwoven web having superabsorbent particles dispersed therein.

These wound care articles have proved very successful in practice, since they are capable of taking up exudates even from great depth, and thus of appreciably improving wound healing especially with chronic and/or edema-related wounds.

However, these wound care articles as such are useful only for relatively flat wounds, but not for deep wounds which, at the floor, have a high potential of exudates.

The term “exudate” refers to any fluid derived from blood plasma via the inflammatory processes of a wound edema. Just like blood is responsible for the transportation of nutrients and other messenger substances and hence for supplying various parts of the body, exudate performs a very similar function in supplying the wound bed and the healing processes taking place therein. To perform this multiplicity of functions, exudate contains a broad spectrum of components, which results in a specific density slightly above that of water. This distinguishes exudate from transudate, which derives from non-inflammatory processes and has a distinctly lower specific density coupled with a low cell and protein content. In addition to providing nutrients for the fibroblasts and epithelial cells, exudate coordinates the various processes of wound healing in a temporal and spatial manner by virtue of its high content of growth factors and cytokines These are formed in particular by thrombocytes, keratinocytes, macrophages and fibroblasts. They influence the motility, migration and proliferation of the various cells involved in wound healing. Thus, the immigration of cells into the wound floor is promoted, as is the supply of the newly formed granulation tissue by angiogenesis. Exudate also augments wound cleaning. Exudate contains various serine, cysteine and aspartate proteases and also matrix metalloproteases, the effect of which is strictly regulated and which degrade not only existing but also newly formed collagen in the wound.

Constituents of physiological exudate are, in particular, salts, glucose, cytokines and growth factors, plasmaproteins, proteases (in particular matrix metalloproteases), granulocytes and macrophages.

A wound is said to be a chronic wound if within a few weeks there is no distinct progression along the wound healing cascade in accordance with the various phases of wound healing. However, exudative phases lasting longer than just three days are considered to be a complication and are said to be a pathological exudation which can contribute to wound chronification. The underlying causes are usually complex and may even well be systemic in nature. However, it is no surprise that, owing to the above-explained significance of exudate for wound healing, complications of wound healing are reflected in a distinctly changed composition and effect for the exudate.

A shift in the concentrations of the individual constituents of the exudate is one reason why the normally cure-promoting exudate loses its positive effect in the case of chronic wounds. Pathological exudate has in particular a significantly raised level of inflammatory cytokines and proteases. There is a reduced level of growth factors, by contrast. A particularly serious difference is due to the activity of the aforementioned matrix metalloproteases. In addition to preparing the wound bed, they are also involved in the later conversion of granulation tissue into scar tissue. These enzymes are normally formed as an inactive pre-enzyme and are regulated in their activation by corresponding inhibitors (tissue inhibitors of metalloproteases, TIMPs), which themselves also have a positive effect on cell growth. This regulatory system appears to be disturbed in a chronic exudate in that the activity of the proteases is enhanced, which may contribute to a regression of wound healing. The pathological exudate has become unbalanced with regard to the proportions of its components and has thus strayed from the equilibrium beneficial to wound progression. This gives rise to various complications which contribute to further deterioration and chronification of the wound.

Exudate removal from a wound edema is thus an essential prerequisite in order that the wound caused by the edema may be cured.

The problem addressed by the present invention is that of providing a wound care article useful in particular for the management of deep wounds.

This problem is solved by the features of the accompanying main claim.

### SUMMARY OF THE INVENTION

Provided herein are systems, methods and compositions for a . . . .

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 shows a wound care article set 10 in a position of application to a deep wound 11, whose wound floor 12 is where exudate 13 collects.

FIG. 2 shows a similar wound care set to FIG. 1 except that the wound inserts 25, 15 are formed without cover.

FIG. 3 shows a similar wound care set to FIG. 1 except that wound inserts comprising superabsorbent fibers are provided here. These are in tampon form for example and are formed without cover. As regards the advantages of this configuration, the description is referenced.

FIG. 4 shows a similar wound care set to FIG. 1 except that here different geometries are shown by way of example for the wound inserts, namely cylindrical (41), shell-shaped (42), tetrahedrally or pyramidally shaped (43) or tamponadelike (44).

FIG. 5 shows a further configuration whereby the wound care set includes not only the first wound care article 54 but also at least one second wound care article 55 which has a segmentable configuration. In this case, the second wound care article is for example made equal to or even larger in terms of area than the first wound care article.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Accordingly there is provided a wound care article set comprising one or more than one first planar wound care article comprising hydroactive polymers. Said set further comprises one or more than one second wound care article comprising hydroactive polymers, wherein the second and any further wound care article has a lower liquid retention and/or a lower liquid uptake capacity than the first wound care article.

The term “hydroactive polymers” is to be understood hereinafter as referring to polymers capable of binding large amounts of liquid. Said polymers preferably comprise superabsorbent substances comprising polyacrylates, modified cellulose and/or alginates.

Liquid uptake capacity and liquid retention refer to the ability of an absorbent material to take up liquids and bind/retain them, respectively. It is generally expressed in grams of a liquid (for example distilled water or 0.9% saline) per gram of absorbent material. Both parameters are a function of the properties of the hydroactive polymer, of its proportion in relation to the overall product and also of the chemical and physical makeup of the overall product.

Various configurations are conceivable for these parameters:

liquid uptake (g/g) within a given interval (15 min, for example);

liquid uptake (g/g) within a given interval while a pressure (0.3 psi, for example) is applied to the absorbent material;

liquid retention (g/g) of an absorbent material saturated by immersion or addition;

liquid retention (g/g) of an absorbent material saturated by immersion or addition while a pressure (0.5 psi, for example) is applied to the saturated absorbent material.

Methods to determine said parameters have been described, for example in U.S. Pat. No. 4,215,692.

This difference in liquid retention and/or liquid uptake capacity is a contributory reason why the present set of wound care articles is capable of using the second and any further wound care articles (wound inserts) to actively acquire liquid, preferably exudate, from the wound floor and then gradually release it to the first wound care article (the wound dressing), which has a relatively higher liquid retention and/or liquid uptake capacity. This prevents any potentially disadvantageous overmoistening of the wound care articles in the wound floor, yet nonetheless ensures effective removal of liquid, preferably exudate, from deep down.

Preferably, a wound dressing is concerned in the case of the first planar wound care article, while a wound insert is concerned in the case of the second and any further wound care article.

Further preferably, the wound inserts, viz., the second and further wound care articles, are divisible into two or more groups differing in liquid retention and/or liquid uptake capacity.

The different liquid retentions and/or liquid uptake capacities give rise to a retention and/or capacity gradient to ensure effective further transportation of liquid in the second and further wound care articles, viz., from the wound care articles of relatively lower liquid retention and/or liquid uptake capacity to the wound care articles of relatively higher liquid retention and/or liquid uptake capacity. The liquid is then finally released to the first wound care article (the wound dressing) which, as already noted above, preferably has the relatively highest liquid retention and/or liquid uptake capacity.

In a further preferred embodiment, the first planar wound care article is larger in terms of area than the second and any further wound care article present.

This makes the second and any further wound care articles present particularly fit for their purpose as wound insert. They can be configured, for example, in the form of dominolike portions (popularly also referred to as “chips” or “pellets”); and they may be formed with or without cover. Other possible embodiments take the form of tamponadelike rolls, cylinders, prisms, tetrahedra or cuboids.

In a further preferred embodiment, the second wound care article and also any further wound care articles have a segmentable configuration. In this case, the second wound care article may be made equal to or even larger in terms of area than the first wound care article. Such a second or further wound care article has, for example, imprinted cutting lines or introduced perforations to enable the second or further wound care article to be subdivided into smaller segments by a carer for example which are then used as wound inserts. In this embodiment, the second wound care article and also any further wound care article is preferably devoid of any cover.

In a further preferred embodiment, the first planar wound care article has a planar portion comprising absorbent material and also a liquid-pervious cover, wherein further the planar portion comprising absorbent material, when viewed plan to its flat side, has an area (F1) which, in the unwetted state of said portion of absorbent material, is from 3% to 75% smaller than the lay-flat area (F2) of the cover. The interspace thus achieved between the cover and the portion of absorbent material is also referred to as an “expansion margin”. This ensures that the portion of absorbent material taking up liquid can expand in volume and is not restricted by the cover.

In a particularly preferred embodiment, the cover when viewed plan to its flat side displays a circumferential protrusion projecting beyond the seam and the absorbent body is free of harsh, sharp edges and corners.

The wound-remote side of the wound care article may similarly be provided with a liquid-impervious backsheet underwear protector.

It may further be provided that at least one of the two wound care articles, preferably the first wound care article, includes a portion comprising a fibrous nonwoven web, preferably an airlaid fibrous nonwoven web.

In a further preferred embodiment, superabsorbent polymers (SAPs) are concerned in the case of said hydroactive polymers in the first wound care article and/or the second and also any further wound care articles.

In a further particularly preferred embodiment, the superabsorbent polymers in the second wound care article and also any further wound care articles are at least partly superabsorbent fibers (SAFs).

Superabsorbent fibers in their pure form generally are devoid of any wicking effect; that is, once liquid has been taken up it is not actively forwarded along the fibers, but leads to gel formation directly at the site of uptake. However, such a wound care article (a wound insert) can be backed with a further wound care article having a higher level of liquid retention and/or liquid uptake capacity to create a retention and/or capacity gradient to ensure that the former wound care article very largely releases the liquid uptake to the latter. This ensures even without any wicking effect that the liquid is transmitted from the wound floor to ultimately the first planar wound care article (i.e., the wound dressing). Said fibers preferably take the form of a fibrous nonwoven web, more preferably in admixture with other fibers (polymer fibers or cellulose).

Since superabsorbent fibers and/or products comprising same generally have a lower level of retention and/or capacity than, for example, fibrous cellulose nonwoven webs admixed with granules of superabsorbent polymers, it is particularly the combination in which the former act as wound inserts and the latter, having a relatively higher level of retention and/or capacity, as wound dressing, which is particularly preferred. The remarkable softness of many superabsorbent fibers is also exploited in this context in that it permits low-pain lining of the wound floor and also, by virtue of the gel formation, an atraumatic changing of the wound inserts.

In a further particularly preferred embodiment, the second wound care article and also any further wound care articles include a coverless portion comprising absorbent material. This is sensible in particular when the hydroactive polymers in the second wound care article and also any further wound care articles take the form of fibers, for example superabsorbent fibers or carboxymethylcellulose fibers. Said fibers, optionally with further types of fibers, may take the form of, for example, fibrous nonwoven webs or of woven or knitted fabrics, in which case it is ensured that they will not depart the composite on wetting and swelling.

This may further be sensible when the superabsorbent polymers are in a firmly immobilized state in the second wound care article and also any further wound care articles, for example in a fibrous nonwoven web matrix, in which case it is ensured that they cannot detach out of this matrix on wetting and swelling.

In a further particularly preferred embodiment, the second wound care article and also any further wound care articles comprise a liquid-conducting material. This can be used to improve the liquid transportation from the wound inserts to the wound dressing—as also supported by the different levels of liquid retention and/or absorption.

Preferably liquid-conducting fibers, such as mercerized fibers, hydrophobic fibers or so-called microfibers, for example:

polyester fibers, e.g., Trevira Finesse, Diolen Soft, Fortrel Microspun, DuPont Micromattique, Primabelle and/or Shingosen;

nylon fibers, such as nylon-6,6, Timbrelle, Supplex Microfiber, Tactel® Micro and/or Silky Touch;

acrylic fibers, such as Microsupreme; and/or

polyurethane fibers.

In a further preferred embodiment, the first and/or second and also any further wound care articles comprise at least one agent selected from the group consisting of:

hemostatic agents, preferably collagen or gelatin;

antimicrobial agents, preferably chitosan, silver or silver ions, copper or copper ions;

agents that inhibit matrix metalloproteases (MMP);

wound healing promoter agents, such as hyaluronic acid;

lactic acid, lactic acid bacteria and/or pre- or probiotic materials.

There is further provided a wound care device for treating wounds using negative atmospheric pressure in the wound region, comprising:

at least a wound-covering element;

at least a negative atmospheric pressure generator means which is optionally attachable to the wound-covering element; and also

at least a wound care article set as claimed in any preceding claim.

Wound care devices of this type—except the wound care article set as claimed in any preceding claim—are known, for example from the commonly assigned patent applications WO2006048246, WO2006056294, WO2006048240 and DE 10 2011 050 047.2, which relate to negative wound pressure therapy (NWPT) and are hereby fully incorporated herein by reference.

The combination of the vacuum wound care devices disclosed in the cited applications with the wound care article set discussed herein offers a whole series of advantages. For instance, it greatly facilitates the acquisition of wound fluids in the wound floor, and the therapeutic efficiency of NWPT, the ultimate purpose of which is to remove wound fluids accumulated in the wound floor, is greatly enhanced.

The invention further provides for the use of a wound care article set and/or a wound care device as claimed in any preceding claim for the treatment of deep wounds.

It is further optionally provided that at least one wound care article of the set also includes an at least portionally liquid-pervious cover.

The latter surrounds the absorbent body, forms a barrier against solid excretions and enables other eliminated substances to pass through to a portion of absorbent material that is disposed within the cover. The cover is preferably at least partly closed off with a seam.

The pores or meshes in the cover are preferably from 0.05 mm to 1.0 mm and more preferably from 0.20 mm to 0.50 mm in size. Mean pore size may be provided in principle to be lower than the mean size of the beads comprising hydroactive polymers. The pores or meshes may further be preferably bounded by the thread or fiber portions which, considered in section through the cover, are approximately arcuate with their arc vertices pointing out.

The covering is preferably formed of woven or nonwoven manufactured fibers, such as polypropylene or polyethylene fibers, but also cotton, silk, or viscose. The covering preferably consists of one from a woven or fibrous nonwoven web fabric having at least an area-specific weight of 20 g/m².

The covering may further consist of a silicone material, for example a silicone lattice or a perforated silicone foil, or a siliconized material of, for example, a lattice or a perforated foil.

Preferably, the cover consists of hydrophobic material, and/or the cover material has a hydrophobic finish. The hydrophobic properties of the cover prevent sticking to the wound surface and contribute to faster passage of the wound exudate particles into the interior of the cover.

It may be provided in this connection that at least a portion of the cover comprises an elastic material, for example fibers composed of Lycra or elastane. This ensures that the portion of absorbent material taking up liquid can expand in volume and is not restricted by the cover.

Absorbent bodies of the type mentioned have been disclosed, for example in commonly assigned WO03094813, WO2007051599 and WO0152780.

The texture of the material of the cover can be such that the cover has a rough inside surface and a smooth outside surface. The rough inside surface of the cover is preferably formed by cone-shaped perforations which each taper in the direction of the inside surface and end in a “protrusion” forming a free edge to the opening. This rough inside surface works to resist displacement of the contents of the cover, so fixing via dots of adhesive can be eschewed. Accordingly, the smooth outside surface of the cover material may be formed by vaulted portions of material which extend between the perforations. A cover material of this type may be called “three-dimensional”, in contradistinction to a cover material that is planar on both sides, and is known, for example from commonly assigned DE102006017194, the disclosure of which is hereby fully incorporated herein by reference.

It is particularly preferable for said three-dimensional cover material to be laminated onto the aforementioned fibrous nonwoven polypropylene web. Improved properties of liquid uptake are a consequence of such a configuration.

The wound-remote side of the wound care article may similarly be provided with a liquid-impervious backsheet underwear protector.

Superabsorbent polymers (SAPs) are manufactured polymers capable of imbibing liquids to a multiple—up to 1000 times—of their own weight. Chemically, they comprise a copolymer of acrylic acid (propenoic acid, C₃H₄O₂) and sodium acrylate (sodium salt of acrylic acid, NaC₃H₃O₂), wherein the ratio between the two monomers may vary. A so-called core-crosslinker (CXL) is additionally included in the monomer solution to join the resultant long-chain polymeric molecules together in places by means of a network of chemical bridges (known as “crosslinks”). These bridges render the polymer insoluble in water. On ingress of water or aqueous solutions of salt, the polymer bead swells up and causes this network of crosslinks to tauten at a molecular level, so the water is no longer able to escape unaided. The superabsorbent polymers may be present in the wound care article of the present invention in the form of a granular material, in the form of a powder, in the form of a loose aggregation, in the form of a compacted aggregation, in the form of a foam, in the form of fibers, in the form of a fibrous knit, laid or nonwoven web fabric and/or a fibrous wadding.

Alternatively, the chosen superabsorbents may be methylacrylic acid based, polyvinyl alcohol-maleic anhydride copolymers, polysaccharide-maleic anhydride copolymers, maleic acid derivatives, acrylamidopropanesulfonic acid copolymers, starch-acrylonitrile graft polymers, gelatinized starch derivatives, alkyl- or hydroxyalkylcellulose, carboxymethylcellulose, starch-acrylic acid graft polymers, vinyl acetate-acrylic ester copolymers, acrylonitrile copolymers or acrylamide copolymers.

Modified cellulose preferably comprises derivatives of cellulose, preferably sulfoalkylated cellulose and derivatives thereof, preferably celluloseethyl sulfonates, carboxyalkylated cellulose, preferably carboxymethylcellulose, carboxyethylcellulose and/or carboxypropylcellulose, more complex cellulose derivatives, such as sulfoethylcarboxymethylcellulose, carboxymethylhydroxyethylcellulose, hydroxypropyl-methylcellulose, and amidated cellulose derivatives, such as carboxymethylcellulose amide or carboxypropylcellulose amide. Carboxymethylcellulose takes the particular form of sodium carboxymethylcellulose and is commercially available under the name of “Hydrofaser”. In hygiene and wound products, the fibers are converted into a planar matrix. As they take up liquid from the wound exudate, the fibers are gradually transformed into a gel pad wherein the liquid is held and not reemitted. The construction of the fibers in question is such that the wound exudate is only taken up in the vertical direction. As a result, the exudate will not flow over the wound edge as long as there is sufficient capacity. This is an effective way to prevent wound edge maceration.

Said hydroactive polymers may also comprise alginates. Alginates are obtained from brown algae and processed into a fibrous nonwoven web. Chemically, alginates are polysaccharides, specifically calcium and/or sodium salts of alginic acids. Alginates are capable of absorbing up to 20 times their own weight of liquid, the wound exudate being imported into the void spaces. The Ca2+ ions in the alginate lattice are exchanged for the Na+ ions from the exudate until the alginate has reached its point of saturation with sodium ions. In the process, the wound dressing swells up and the alginate fiber is transformed into a gel body as a result of the fibers swelling up.

Said hydroactive polymers may similarly also comprise hydrogel nanoparticles comprising hydroxy-terminated methacrylate monomers, such as 2-hydroxyethyl methacrylate (HEMA) and/or 2-hydroxypropyl methacrylate (HPMA), which are marketed as Altrazeal, for example.

It is particularly preferable for at least one wound care article of the set to further comprise a fibrous nonwoven web comprising cellulose fibers which hereinafter is also referred to as an absorbent body.

The absorbent body may preferably comprise an essentially flat absorbent body which is made of an absorbent material and which consists of an imbibing fibrous nonwoven web incorporating superabsorbent polymers dispersed therein. These may be present in the form of a granular material, in the form of a powder, in the form of a loose aggregation, in the form of a compacted aggregation, in the form of a foam, in the form of fibers, in the form of a fibrous knit, laid or nonwoven web fabric and/or a fibrous wadding.

The absorbent body in question comprises at least one material selected from the group containing a mat, in particular fibrous nonwoven web airlaid from said yarns or fibers of superabsorbent polymers having incorporated superabsorbent polymers, and/or a loose filling of superabsorbent polymers. Said airlaid mat may preferably include an essentially flat portion of absorbent material, said portion of absorbent material consisting for example of an imbibing fibrous nonwoven web formed from the fibers mentioned and having superabsorbent polymers dispersed therein.

This absorbent body may correspond to the absorbent insert that is present in an assignee wound dressing as for example disclosed in WO03094813, WO2007051599 and WO0152780 and as marketed under the trade name “sorbion sachet”. The disclosure of the cited documents is hereby fully incorporated in the disclosure of this document by reference.

The absorbent body in some other configuration may similarly form a core which comprises—optionally flocklike—fibers or yarns of superabsorbent polymers and also superabsorbent polymers in granule form, in which case the granules are adhered and/or fused to the fibers/yarns at two or more heights, and the granules are distributed across more than 50% of the entire design height of a portion of the core at least, in which case there are mingled regions of granules and fibers. The weight fraction of superabsorbent polymers here may preferably be in the range between 10-25 wt %. Similar designs are known from conventional incontinence materials and like sanitary napkins are known for their cushioning properties. A cover may be disposed around said core in an overlapping arrangement in regions, and which for example conceals an adhered seam and/or is part thereof.

It is particularly preferable for the absorbent body to comprise a fibrous web, preferably a nonwoven or airlaid web which consists of superabsorbent fibers (“SAFs”, preferably polyacrylates) or contains same as a constituent part. The fibers may for example be blended with fluff pulp (cellulose) or with polyester fibers. A layered construction may be provided as an alternative or in addition.

The absorbent body in some other configuration may similarly contain at least one flat ply comprising superabsorbent-polymer fibers or yarns having superabsorbent polymers adhered in granule form. This, in a preferred configuration, results in a construction for the body where there are at least two layers in that at least one toplayer puts a layer comprising superabsorbent polymers underneath. A second, flanking toplayer may optionally be provided.

All this without there being any mingling between fibers and superabsorbent polymers in the plane; but merely fixed adjacencies between the two materials. The optionally provided plurality of plies may in one preferred configuration also be physically compacted together by rolling, pressing, calendering or similar processes.

It is particularly preferable for said absorbent body to have an area dimension of 5×10, 5×20, 10×20, 10×10, 10×15 or 15×15 cm.

The basis weight in this case may be in the range between ≧50 and ≦2000 g/m². Preference is here given to basis weights of 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, and/or 2000 each +/−25 g/m².

Thickness may here be in the range between ≧2 and ≦50 mm. Preference is given to thicknesses of 2, 4, 6, 8, 10, 12, 14, 16, 18, 20, 22, 24, 26, 28, 30, 32, 34, 36, 38, 40, 42, 44, 46, 48, and/or 50 each +/−1 mm.

The uptake capacity may here be in the range between ≧3 and ≦30 ml of 0.9% saline/m² at 0.2 psi pressure. Preference here is given to values of 3, 4, 5, 6, 7, 8, 9, 10, 11, 12, 13, 14, 15, 16, 17, 18, 19, 20, 21, 22, 23, 24, 25, 26, 27, 28, 29, and/or 30 ml of 0.9% saline/m². Alternatively, the uptake capacity can be in the range between ≧2 and ≦50 g of water/g. Preference in this case is given to values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 g of water/g.

The overall content of superabsorbent polymers may here be in the range between ≧5 and ≦100% w/w. Preference is here given to values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100% w/w.

Tensile strength may here be in the range between ≧5 and ≦80 N/5 cm. Preference is here given to values of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80 N/5 cm.

Extensibility here can be in the range between ≧10 and ≦80%. Preference is here given to values of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80%.

When superabsorbent fibers are used, the following types have turned out to be particularly advantageous in practice, as shown in Table 1:

TABLE 1

| Type | 1 | 2 | 3 | 4 | 5 | 6 |
|---|---|---|---|---|---|---|
| construction 1 | layered | 40% | bicomponent | layered | 25% polyester; | 40% polyester |
| | construction: | polyester | fiber between | construction: | 75% SAF | chopped fiber; |
| | thermobonded | chopped | SAF and a | thermobonded | | 60% SAF |
| | airlaid with | fiber; 60% | thermoplastic | airlaid with | | |
| | laminated | SAF | | laminated | | |
| | nonwoven | | | nonwoven | | |
| construction 2 | bicomponent | needlefelt | carded | bicomponent | needlefelt | needlefelt |
| | fiber between | | thermobonded | fiber between | | |
| | SAF and a | | nonwoven | SAF and a | | |
| | thermoplastic + | | | thermoplastic + | | |
| | fluff pulp | | | fluff pulp | | |
| type of SAF | 101/6/10 | 102/52/10 | 102/52/10 | 101/6/10 | | |
| fiber | | | | | | |
| weight (g/m²) | 560 | 540 | 1000 | 350 | 150 | 380 |
| thickness (mm) | 6 | 5.4 | 20 | 3.5 | 2.4 | 3.8 |
| uptake capacity | 31.21 of | >20 g of | >16 g of | 19.5 1 of | >25 g of 0.9% | >17 g of |
| | water/m² | water/g | water/g or | water/m² | saline/g | water/g or |
| | | | 16 000 g of | | | 6400 g/m² |
| | | | water/m² | | | |
| uptake capacity | 16 | | | 16 | | |
| under pressure | | | | | | |
| (ml of 0.9% | | | | | | |
| saline/m² at | | | | | | |
| 0.2 psi | | | | | | |
| pressure) | | | | | | |
| overall content | 18 | 40 | 50 | 18 | 75 | 60 |
| of | | | | | | |
| superabsorbent | | | | | | |
| polymer (% | | | | | | |
| w/w) | | | | | | |
| tensile strength | | | | | 16 ± 13 | 16 ± 13 |
| (N/5 cm) | | | | | | |
| extensibility | | | | | 60 ± 18 | 60 ± 18 |
| (%) | | | | | | |

Similarly preferred parametric ranges as recited above apply. Liquid retention can be between ≧5 and ≦100 g/g. Preference in this case is given to values of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 g/g

The following types have turned out to be particularly advantageous in practice, as shown in Table 2:

TABLE 2

| Type | 1 | 2 | 3 | 4 | 5 | 6 | 7 | 8 |
|---|---|---|---|---|---|---|---|---|
| weight (g/m²) | 450 | 300 | 150 | 50 | 100 | 120 | 140 | 440 |
| thickness (mm) | 1.3 | 1.2 | 0.9 | 0.7 | 0.7 | 0.76 | 1 | 1.2 |
| liquid retention (g/g) | 28 | 33 | 28 | 15 | 25 | 28 | 11.5 | 38 |
| tensile strength (N/5 cm) | 25 | 55 | 20 | 20 | 20 | 20 | 15 | 20 |
| uptake capacity (g/g) | 45 | 20 | 50 | 20 | 40 | 50 | 28 | 55 |

The following types have turned out to be particularly advantageous in practice:

Furthermore, a wound care article of the set and/or an absorbent body contained therein may display repetitive patternings or grains, for example a diamond pattern, a punched pattern or the like.

It is further particularly preferable for said absorbent body to include not only a ply comprising superabsorbent polymers but also at least one second flanking ply that contains little if any by way of superabsorbent polymers and areawise extends beyond the first ply mentioned. This ensures that the ply comprising superabsorbent polymers can increase in volume in accordance with the liquid uptake without the increase in volume being externally visible, since the latter is hidden by the second layer.

In a further preferred embodiment, the wound care article set further comprises a wound-distancing lattice.

The term “wound-distancing lattice” refers to a lattice- or gauzelike structure which—frequently as a so-called “primary dressing”—is placed directly on a wound before optionally a secondary dressing is applied. Wound-distancing lattices are frequently also referred to as “wound gauzes” and serve primarily to prevent sticking of the overall wound dressing to the wound. For this purpose, wound-distancing lattices are fabricated of or coated with a material that reduces/prevents any adherence to the wound. Alternatively, the geometry of the wound-distancing lattice can also be configured such that adherence to the wound is reduced/prevented.

Commercially available examples of wound-distancing lattices include for instance the products “Mepitel” (silicone-coated, elastic nylon mesh), “Physiotulle”, “Urgotül” (mesh of polyester fibers drenched with hydrocolloid particles and white petroleum jelly) and “ADAPTIC” (smooth knit of viscose, impregnated with an oil-in-water emulsion).

The aforementioned products comprise a two-dimensional wound-distancing lattice, which thus essentially assume the form of a gauze or of a mesh. A three-dimensional wound-distancing lattice is known under the name “sorbion plus” and described in particular in commonly assigned EP2004116, the content of which is hereby fully incorporated herein by reference.

Such a three-dimensional wound-distancing lattice includes a liquid-pervious conformable portion which is composed of a thermoplastic material and has a first, smooth surface and a second, rough surface remote from the first, smooth surface. The second, rough surface is created by a multiplicity of three-dimensional perforations, the walls of which protrude from the first, smooth surface and each end in a protruding rim having a free edge, so they endow the second surface with a rough grip.

The perforations are produced by one-sidedly inserted punching or by thermoforming over a foraminous sheet. This creates a rough side with protrusions and also a more or less smooth side.

A wound-distancing lattice is reliable in preventing the wound care article from sticking to the wound and wound edge maceration. Therefore, unlike with the foams known from the prior art, there is no longer any need for the active element in question to be cut to size to match the shape of the wound, greatly reducing the expenditure of labor on the part of the medical personnel.

It is further preferable for at least one wound care article to have a cover which in turn has means engineered and/or selected such that the cover is at least partly deformable in a purposive manner via the absorbent body increasing in volume due to liquid uptake. In principle, such behavior can be achieved with unidirectionally elastic material, i.e., a material which is extensible in some direction but not in the direction orthogonal thereto.

It is further provided that at least one wound care article comprises a composition containing at least one active nutritive, at least one active disinfectant/decontaminant and/or at least one active protease inhibitor ingredient and/or ingredient complex for external management and/or treatment of wounds to the human or animal body.

The present invention is more particularly elucidated by the figures shown and discussed hereinbelow. It must be noted in this connection that the figures are merely descriptive in character and are not intended to limit the invention in any form.

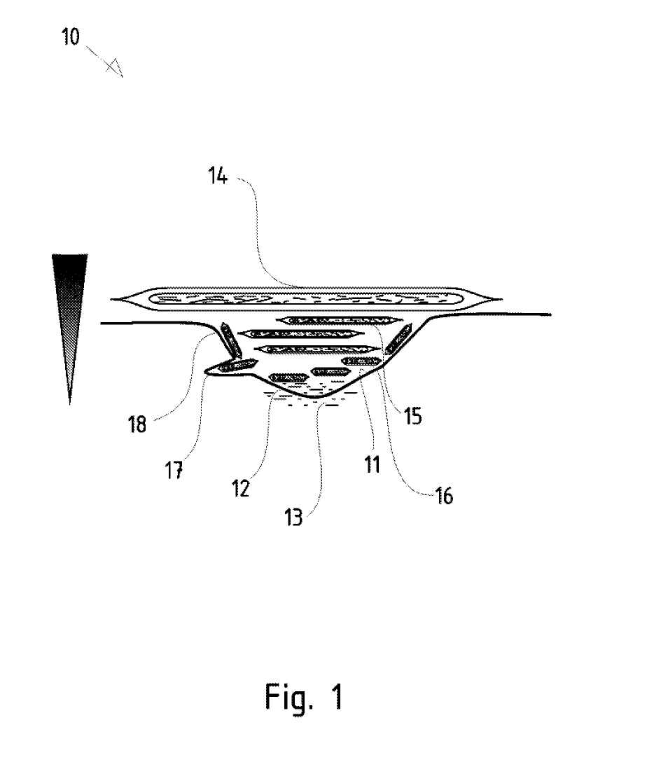

FIG. 1 shows a wound care article set 10 in a position of application to a deep wound 11, whose wound floor 12 is where exudate 13 collects. The set comprises a first planar wound care article 14 comprising hydroactive polymers and also further wound care articles 15, 16 comprising hydroactive polymers. The further wound care articles 15, 16 have a lower level of liquid retention and/or a lower level of liquid uptake capacity than the first wound care article 14. A wound dressing is concerned in the case of the first planar wound care article, while a wound insert is concerned in the case of the second and any further wound care article.

FIG. 1 shows a preferred embodiment, which features two groups of wound inserts differing in liquid retention and/or liquid uptake capacity, those of relatively lower retention and/or capacity being preferentially disposed in the vicinity of the wound floor, as shown in FIG. 1 and as symbolized by the gradient indicated by the gray-scale value wedge at left in said figure. The different liquid retentions and/or liquid uptake capacities give rise to a retention and/or capacity gradient to ensure effective further transportation of liquid in the second and further wound care articles, viz., from the wound care articles of relatively lower liquid retention and/or liquid uptake capacity to the wound care articles of relatively higher liquid retention and/or liquid uptake capacity. The liquid is then finally released to the first wound care article (the wound dressing) which, as already noted above, preferably has the relatively highest liquid retention and/or liquid uptake capacity. Said wound inserts are also very useful as fillers for wound pockets 17 or for lining the wound edge 18.

The wound dressing 14 shown in FIG. 1 is a product comprising superabsorbent polymers in a fibrous nonwoven cellulose matrix having a spaced-apart cover formed of a fibrous nonwoven polypropylene web. The grammage of the product, which is commercially available under the name “sorbion sachet” for example, was chosen so as to achieve a high capacity and/or retention. The wound dressing further comprises the expansion margin described above.

The wound inserts 15, 16 shown in FIG. 1 likewise consist of a product comprising superabsorbent polymers in a fibrous nonwoven cellulose matrix having a spaced-apart cover formed of a fibrous nonwoven polypropylene web.

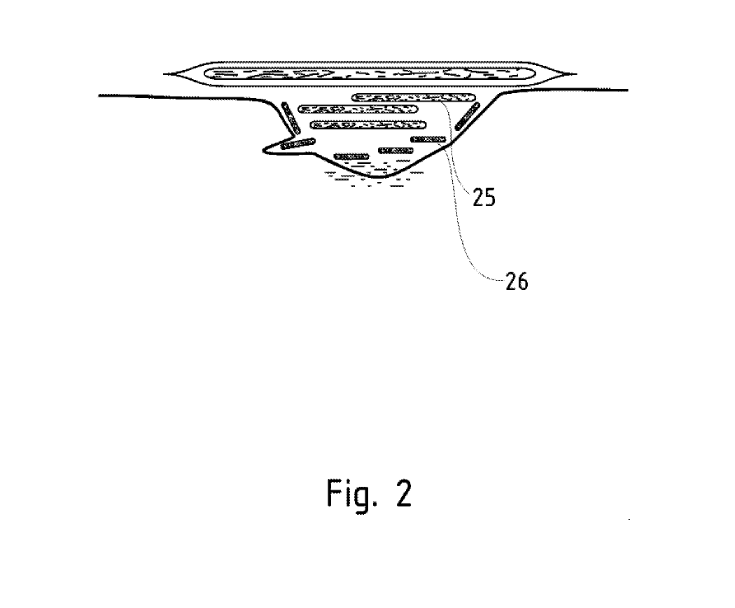

FIG. 2 shows a similar wound care set to FIG. 1 except that the wound inserts 25, 15 are formed without cover.

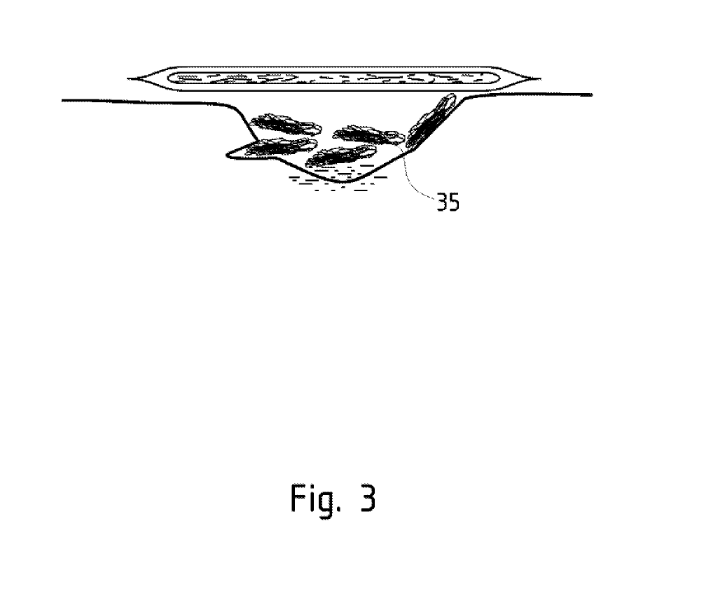

FIG. 3 shows a similar wound care set to FIG. 1 except that wound inserts comprising superabsorbent fibers are provided here. These are in tampon form for example and are formed without cover. As regards the advantages of this configuration, the description is referenced.

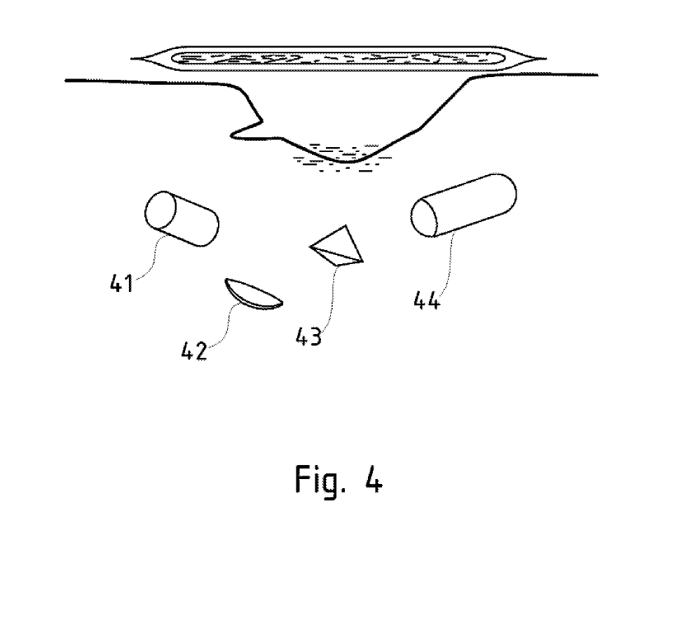

FIG. 4 shows a similar wound care set to FIG. 1 except that here different geometries are shown by way of example for the wound inserts, namely cylindrical (41), shell-shaped (42), tetrahedrally or pyramidally shaped (43) or tamponadelike (44).

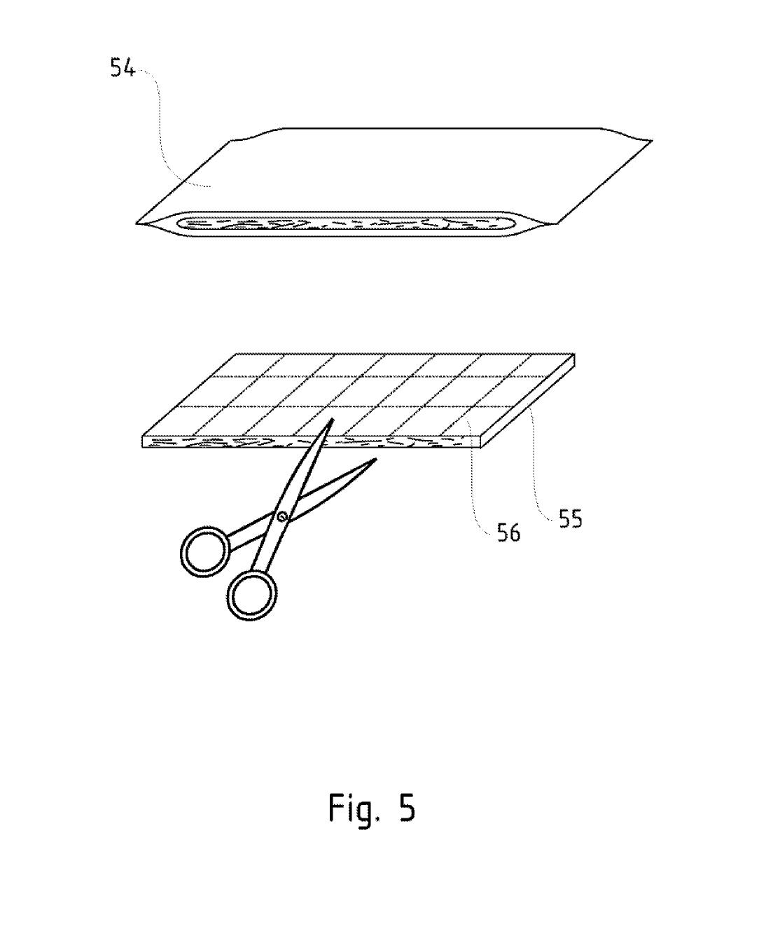

FIG. 5 shows a further configuration whereby the wound care set includes not only the first wound care article 54 but also at least one second wound care article 55 which has a segmentable configuration. In this case, the second wound care article is for example made equal to or even larger in terms of area than the first wound care article.

The second wound care article has imprinted cutting lines 56 to enable the second wound care article to be subdivided into smaller segments by a carer for example which are then used as wound inserts. In this embodiment, the second wound care article and also any further wound care article is preferably devoid of any cover. Alternatively, the second wound care article may also have introduced perforations.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A wound care article set comprising: at least a first planar wound care article comprising hydroactive polymers, further comprising at least one second wound care article comprising hydroactive polymers, wherein the second wound care article has a lower liquid retention and/or a lower liquid uptake capacity than the first wound care article.

2. The wound care article set as claimed in claim 1, wherein the first planar wound care article is a wound dressing, while the second wound care article is a wound insert.

3. The wound care article set as claimed in claim 2, wherein the wound insert of the second wound care article is divisible into two or more groups differing in liquid retention and/or liquid uptake capacity.

4. The wound care article set as claimed in claim 3, wherein the first planar wound care article is larger in terms of area than the second wound care article.

5. The wound care article set as claimed in claim 4, wherein the second wound care article have a segmentable configuration.

6. The wound care article set as claimed in claim 5, wherein the first planar wound care article has a planar portion comprising an absorbent material and a liquid-permeable cover, wherein further the planar portion comprising absorbent material, when viewed plan to its flat side, has an area (F1) which in the unwetted state of said portion of absorbent material, is from 3% to 75% smaller than the lay-flat area (F2) of the liquid-permeable cover.

7. The wound care article as claimed in claim 6, characterized in that at least one of the first or second wound care articles includes a portion comprising a fibrous nonwoven web, wherein the fibrous nonwoven web is an airlaid fibrous nonwoven web.

8. The wound care article set as claimed in claim 7, wherein the hydroactive polymers of the first wound care article or the second wound care article are superabsorbent polymers (SAPs).

9. The wound care article set as claimed in claim 8, wherein the superabsorbent polymers in the second wound care article are at least partly superabsorbent fibers (SAFs).

10. The wound care article set as claimed in claim 9, wherein the second wound care article includes a coverless portion comprising absorbent material.

11. The wound care article set as claimed in claim 10, wherein the second wound care article comprise a liquid-conducting material.

12. The wound care article set as claimed in claim 11, wherein the first or second wound care article comprise at least one agent selected from the group consisting of: hemostatic agents, preferably collagen or gelatin; antimicrobial agents, preferably chitosan, silver or silver ions, copper or copper ions; agents that inhibit matrix metalloproteases (MMP); wound healing promoter agents, such as hyaluronic acid; lactic acid, lactic acid bacteria and/or pre- or probiotic materials.

13. A wound care device for treating wounds using negative atmospheric pressure in the wound region, comprising: at least a wound-covering element; at least a negative atmospheric pressure generation system that is attachable to the wound-covering element; and at least a wound care article set as claimed in claim 1 operably coupled to the wound covering element.

14. The method of using a wound care article set as claimed in claim 1 for the treatment of deep wounds.
